(19) United States
(12) Patent Application Publication
(10) Pub. No.: US 2023/0201598 A1
(43) Pub. Date: Jun. 29, 2023

(54) MOVEMENT MONITOR SENSOR

(71) Applicant: Industrial Technology Research Institute, Hsinchu (TW)

(72) Inventors: JO-PING LEE, Tainan City (TW); SHENG-HONG TSENG, Taipei City (TW); CHUNG-HSIN SU, Hsinchu City (TW); YUNG-HSIANG WU, Hsinchu City (TW)

(21) Appl. No.: 18/089,203

(22) Filed: Dec. 27, 2022

Related U.S. Application Data
(60) Provisional application No. 63/294,133, filed on Dec. 28, 2021.

(30) Foreign Application Priority Data
Nov. 8, 2022 (TW) 111142544

(51) Int. Cl. A61N 1/36

(52) U.S. Cl. CPC A61N 1/36139; A61N 1/36067

(57) ABSTRACT

A movement monitor sensor includes a body, a conduction area, at least one depth electrode set and a flat electrode set. The body has an axis and two axial ends. The conduction area at one axial end connects a conductive wire. The depth electrode set includes four separate depth electrodes disposed on the body by surrounding the axis and connected individually with first wires. The flat electrode set includes a substrate disposed at another axial end and four separate flat electrodes disposed at the substrate by surrounding the axis and connected individually with second wires. The conductive wire, the first and second wires are individually connected with the conduction area, the depth electrodes and the flat electrodes. When a human movement changes, a processor evaluates impedance variations generated by the depth electrode set, the flat electrode set and/or the conduction area to determine electrical stimulation control upon human brain.

### CROSS REFERENCE TO RELATED APPLICATION

This application claims the benefits of U.S. provisional application Series No. 63/294,133, filed on Dec. 28, 2021, and Taiwan application Serial No. 111142544, filed on Nov. 8, 2022, the disclosures of which are incorporated by references herein in its entirety.

### TECHNICAL FIELD

The present disclosure relates in general to a medical technology, and more particularly to a movement monitor sensor.

### BACKGROUND

Nuclei in the brain area are interconnected through many nerve fibers. By taking the subthalamic neucleus (STN) as an example, in order to produce smooth movements, the STN would generate electrical signals to activate the other nerve nuclei, such as the globus pallidus interna (GPi), such that secretion of dopamine in the putamen would be induced to affect on/off of the corresponding movement. However, if this neural network cannot be activated, then clinical symptoms such as rigidity or tremors due to insufficient dopamine secretion would appear; i.e., typical clinical symptoms of Parkinson's disease.

Major movement impairments of Parkinson's disease include difficulty in initiating movements (such as a movement initiator from a sitting to standing position) and/or difficulty in transitioning between movement states (such as turning around).

In addition to medications, a popular way is to implant neuromodulation probes. This type of probes is provided with a plurality of electrodes, and each of the electrodes is connected to a wire. Through energizing the electrode via the wire, the electrode can be used to activate the neural network by stimulating the STN or the GPi, such that the movement impairments can be improved.

Nevertheless, it has been clinically found that some patients do not respond well to continuous electrical stimulation. Only at the moment while a movement is changing or initiating (for example, at a switching point in a gait cycle such as a transition from sitting to standing or turning, or from a heel contact to an off contact), the instant electrical stimulation be effective to improve walking ability.

Accordingly, how to develop a “movement monitor sensor” that can provide information helpful to determine the timing of electrical stimulation by sensing patient's movement is an urgent issue for those in the relevant technical field.

### SUMMARY

In one embodiment of this disclosure, a movement monitor sensor, disposed in a human brain and connected externally with a processor, includes:

a body, having an axis and two axial ends along the axis and opposite to each other;

a conduction area, disposed at one of the two axial ends, connected with a conductive wire;

at least one depth electrode set, including four depth electrodes disposed on a surface of the body by surrounding the axis, the four depth electrodes being distributed to four directions, each of the four depth electrodes being connected with a first wire; and

a flat electrode set, including a substrate and four flat electrodes, the substrate having oppositely a first surface and a second surface, the second surface being disposed at one of the two axial ends opposite to the conduction area, the four flat electrodes being disposed at the substrate by surrounding the axis to correspond individually the four directions, each of the four flat electrodes being connected with a second wire;

wherein the conductive wire, the first wires and the second wires are individually connected electrically with the conduction area, the depth electrodes and the flat electrodes; wherein, when a movement of a human changes, the processor evaluates impedance variations generated by the at least one depth electrode set, the flat electrode set and/or the conduction area to determine a corresponding electrical stimulation control upon brain tissue of the human.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a schematic perspective view of an embodiment of the movement monitor sensor in accordance with this disclosure;

FIG. 2 is a schematic top view of FIG. 1;

FIG. 3 is a schematic cross-sectional view of FIG. 2 along line A-A;

FIG. 4 is a schematic view showing the sensor of FIG. 1 furnished with a fixing part is implanted into a human brain;

FIG. 5 demonstrates schematically movements of electrodes of FIG. 1 while the human moves horizontally;

FIG. 6 demonstrates schematically movements of electrodes of FIG. 1 while the human moves vertically;

FIG. 7 shows schematically relationships between the brain tissue and the electrodes while the human moves in accordance with this disclosure;

FIG. 8 shows schematically a typical design example of a facing-route synchronous sequential voltage measurement circuit applicable to this disclosure;

FIG. 9A to FIG. 11B show schematically examples of multi-channel pairing switches applicable to this disclosure;

FIG. 12A shows schematically impedance variations while the human rises and tilts forward;

FIG. 12B shows schematically impedance variations while the human walks;

FIG. 13 demonstrates schematically a linear relationship between impedance variations and displacements caused by deformations of the brain tissue while the human moves in one direction in accordance with this disclosure;

FIG. 14 demonstrates schematically a linear relationship between impedance variations and compression caused by deformations of the brain tissue while the human moves in one direction in accordance with this disclosure;

FIG. 15 shows schematically another embodiment of the movement monitor sensor in accordance with this disclosure, and movements of electrodes of this embodiment while the human moves horizontally; and

FIG. 16 demonstrates schematically movements of electrodes of the embodiment of FIG. 15 while the human moves vertically.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

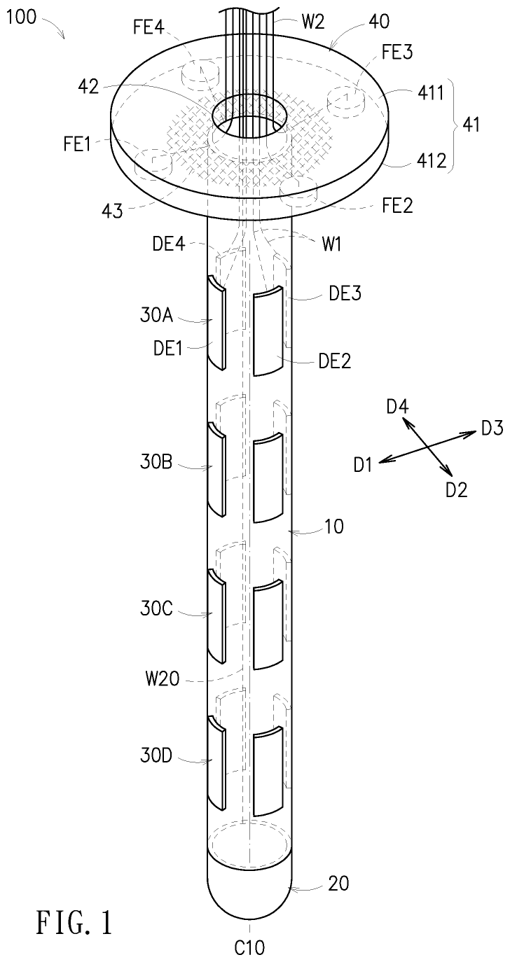

Referring to FIG. 1, a movement monitor sensor 100, to be disposed in a human brain, is suitable to connect a power device and a processor (not shown in the figure). The processor is adapted to control the power device to supply electric currents to the movement monitor sensor 100. According to impedance variations detected by the movement monitor sensor 100, corresponding electric currents to control electrical stimulation upon nerve fibers surrounding the movement monitor sensor 100 can be then determined. In this disclosure, types of the processor are not specifically limited, such as computer devices.

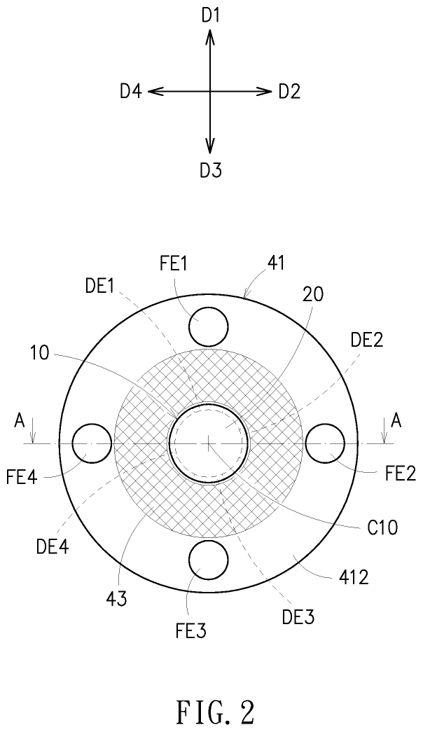

Referring to FIG. 1 to FIG. 3, movement monitor sensor 100 includes a body 10, a conduction area 20, four depth electrode sets 30A˜30D and a flat electrode set 40.

The body 10 has an axis C10, and the conduction area 20 and the flat electrode set 40 are individually disposed to two opposite axial ends of the body 10 with respect to the axis C10.

The conduction area 20, formed by a conductive material, is connected with a conductive wire W20.

These four depth electrode sets 30A˜30D are disposed at the body 10 in parallel to the axis C10. Each of these depth electrode sets 30A˜30D includes a first depth electrode DE1, a second depth electrode DE2, a third depth electrode DE3 and a fourth depth electrode DE4.

The first depth electrode DE1, the second depth electrode DE2, the third depth electrode DE3 and the fourth depth electrode DE4 are disposed on a surface of the body 10 by surrounding the axis C10. In addition, each of the first depth electrode DE1, the second depth electrode DE2, the third depth electrode DE3 and the fourth depth electrode DE4 is connected with a first wire W1.

The first depth electrode DE1, the second depth electrode DE2, the third depth electrode DE3 and the fourth depth electrode DE4 are individually provided by having normally in an outward manner a first direction D1, a second direction D2, a third direction D3 and a fourth direction D4, respectively.

The first direction D1, the second direction D2, the third direction D3 and the fourth direction D4 are individually outward radial lines centered at the common axis C10, and spaced by equal angles. The first direction D1 and the third direction D3 are colinear but extend oppositely to each other with respect to the axis C10, the second direction D2 and the fourth direction D4 are colinear but extend oppositely to each other with respect to the axis C10, and the first direction D1 is disposed between the second direction D2 and the fourth direction D4.

It shall be noted that, in this embodiment, though each of the four depth electrode sets 30A˜30D includes four depth electrodes (i.e., the first depth electrode DE1, the second depth electrode DE2, the third depth electrode D3 and the fourth depth electrode DE4) for performing motion detection or directional electrical stimulation at the corresponding four different directions, yet, in some other embodiments, determination of the number and arrangement of the depth electrodes for each the depth electrode set is not limited thereto, but depends upon practical requirements.

The flat electrode set 40 includes a disc-shape substrate 41 and a first flat electrode FE1, a second flat electrode FE2, a third flat electrode FE3 and a fourth flat electrode FE4. The first flat electrode FE1, the second flat electrode FE2, the third flat electrode FE3 and the fourth flat electrode FE4 are individually disposed at the aforesaid first direction D1, second direction D2, third direction D3 and fourth direction D4, respectively.

It shall be noted that, in this embodiment, though the flat electrode set 40 includes four flat electrodes (i.e., the first flat electrode FE1, the second flat electrode FE2, the third flat electrode FE3 and the fourth flat electrode FE4) for performing motion detection in the corresponding four different directions, yet, in some other embodiments, determination of the number and arrangement of the flat electrodes for the flat electrode set 40 is not limited thereto, but depends upon practical requirements.

The substrate 41 is furnished with a central through hole 42 parallel to the axis C10. In this embodiment, a material for the substrate 41 can be one of silicone or thermoplastic polyurethane (TPU), but not limited thereto.

In addition, a shape or size of the substrate 41 is not specifically limited. As shown in this embodiment, the substrate 41 is shaped to be a ring disc having a maximum diameter D5 substantially equal to or less than 10 mm.

The substrate 41 has oppositely a first surface 411 and a second surface 412. The second surface 412 of the substrate 41 is furnished with a micro structure 43 protruding over the substrate 41 by a height or heights not specifically limited, such as a length equal to or greater than 50 μm.

The first flat electrode FE1, the second flat electrode FE2, the third flat electrode FE3 and the fourth flat electrode FE4 are disposed at the substrate 41 by surrounding the central through hole 42.

The first flat electrode FE1, the second flat electrode FE2, the third flat electrode FE3 and the fourth flat electrode FE4 are corresponding to the first direction D1, the second direction D2, the third direction D3 and the fourth direction D4, respectively. Each of the first flat electrode FE1, the second flat electrode FE2, the third flat electrode FE3 and the fourth flat electrode FE4 is connected with a second wire W2.

The substrate 41 is disposed at one axial end of the body 10 opposite to another axial end having the conduction area 20, by having the second surface 412 thereof to connect the body 10, and by having the central through hole 42 to center the axis C10 of the body 10. Namely, in a bottom view, a center of the hole 42 is located at the axis C10, such that the first flat electrode FE1, the second flat electrode FE2, the third flat electrode FE3 and the fourth flat electrode FE4 at the substrate 41 can surround the axis C10.

All the first wires W1, the second wires W2 and the conductive wire W20 are arranged to pass through the hole 42.

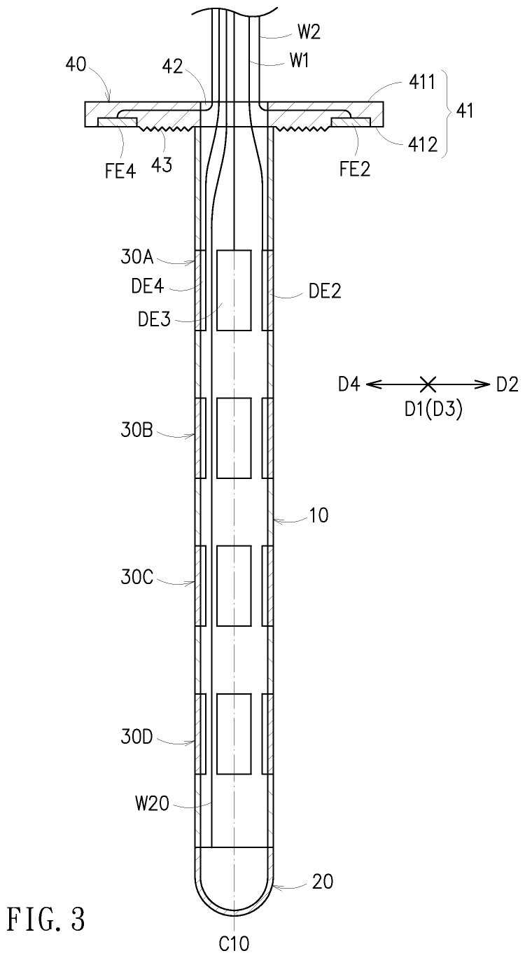

It shall be explained that, in FIG. 1 and FIG. 3, though only the first depth electrode DE1, the second depth electrode DE2, the third depth electrode DE3 and the fourth depth electrode DE4 of the depth electrode set 30A are shown to connect respective first wires W1, yet the other depth electrode sets 30B˜30D are also connected with individual first wires W1, but omitted herein in the figures.

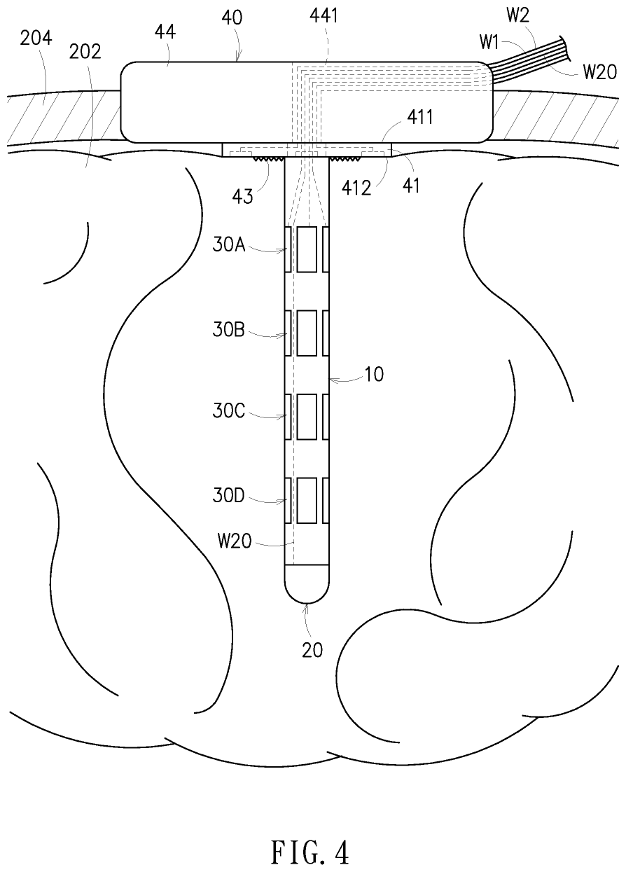

Referring to FIG. 4, the body 10 of the movement monitor sensor 100 is to be implanted into a human brain 202. With the second surface 412 of the substrate 41 of the flat electrode set 40 to approach the brain tissue 202, the micro structure 43 disposed on the second surface 412 can provide direct contact between the substrate 41 and the brain tissue 202, particularly with the meninges to provide adhesion for ensuring the engagement in between.

The first surface 411 of the substrate 41 is provided with a fixing part 44 for disposing at a head bone 204. As shown, the brain tissue 202 is located under the head bone 204. With the fixing part 44 to be disposed at the head bone 204, all the body 10, the conduction area 20, the depth electrode sets 30A˜30D and the flat electrode set 40 would be sealed inside the head bone 204. The fixing part 44 can be made of a rigid material such as a plastics. The fixing part 44 is furnished with a channel 441 for allowing the first wires W1, the second wires W2 and the conductive wire W20 to extend thereinside and to pass therethrough for further protruding out of the fixing part 44 to the exterior of the head bone 204.

Electric currents are introduced to energize the depth electrode sets 30A˜30D, the flat electrode set 40 and the conduction area 20 correspondingly through the first wires W1, the second wires W2 and the conductive wire W20. When the movement is changed, the processor would evaluate impedance variations generated by the depth electrode sets 30˜30D, the flat electrode set 40 and/or the conduction area 20 for the brain tissue 202 to perform the corresponding electrical stimulation control.

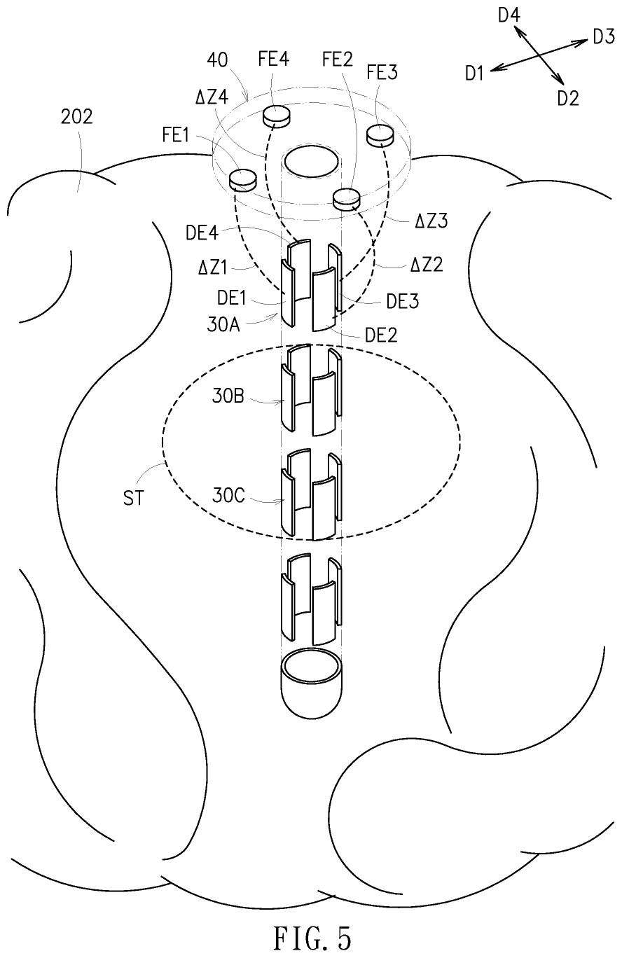

Referring to FIG. 5, when the human moves, electrodes in the flat electrode set 40 (such as the first flat electrode FE1, the second flat electrode FE2, the third flat electrode FE3 and the fourth flat electrode FE4) and the nearest depth electrodes in depth electrode set 30A (such as the first depth electrode DE1, the second depth electrode DE2, the third depth electrode DE3 and the fourth depth electrode DE4) would generate corresponding impedance variations (such as first impedance variations ΔZ1, second impedance variations ΔZ2, third impedance variations ΔZ3 and fourth impedance variation ΔZ4), and then the processor can evaluate multiple impedance variations to control the electrical stimulation ST upon the brain tissue 202.

When the human moves in parallel to the first direction D1 and the third direction D3 (i.e., while moving horizontally back and forth), the first flat electrode FE1 and the nearest first depth electrode DE1_of the depth electrode set 30A would generate a first impedance variation ΔZ1, and the third flat electrode FE3 and the nearest third depth electrode DE3 of the depth electrode set 30A would generate a third impedance variation ΔZ3. The processor would compare the first impedance variation ΔZ1 to the third impedance variation ΔZ3 to determine the corresponding electrical stimulation control for the brain tissue 202, and further to control the two middle depth electrode sets 30B, 30C to perform relevant electrical stimulation ST upon nerve fibers of the nearby brain tissue 202. In this embodiment, electrodes of the depth electrode sets 30B, 30C can be used as stimulating electrodes for executing the electrical stimulation ST.

Similarly, when the human moves in parallel to the second direction D2 and the fourth direction D4 (i.e., while moving left and right), the second flat electrode FE2 and the nearest second depth electrode DE2 of the depth electrode set 30A would generate a second impedance variation ΔZ2, and the fourth flat electrode FE4 and the nearest fourth depth electrode DE4 of the depth electrode set 30A would generate a fourth impedance variation ΔZ4. The processor would evaluate the second impedance variation ΔZ2 and the fourth impedance variation ΔZ4 to determine the electrical stimulation control upon the brain tissue 202, and further to control the two middle depth electrode sets 30B, 30C to perform corresponding electrical stimulation ST to the nerve fibers of the nearby brain tissue 202. In this embodiment, electrodes in the depth electrode sets 30B, 30C can perform as the stimulating electrodes to provide the electrical stimulation ST.

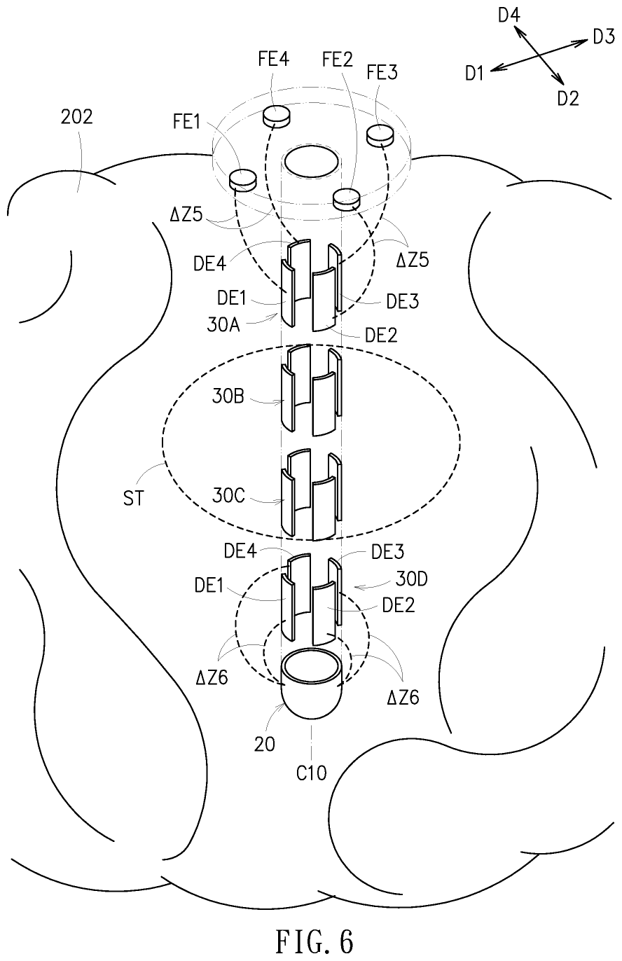

Referring to FIG. 6, when the human moves in parallel to the axis C10 (i.e., while moving up and down vertically), the flat electrode set 40 and the nearest depth electrode set 30A would generate impedance variations. Namely, a fifth impedance variation ΔZ5 would be generated between each of the first flat electrode FE1, the second flat electrode FE2, the third flat electrode FE3 and the fourth flat electrode FE4 and the corresponding nearest one of the first depth electrode DE1, the second depth electrode DE2, the third depth electrode DE3 and the fourth depth electrode DE4 of the depth electrode set 30A, respectively. In addition, the conduction area 20 and the nearest depth electrode set 30D would generate impedance variations. Namely, a sixth impedance variation ΔZ6 would be generated between the conduction area 20 and each of the first depth electrode DE1, the second depth electrode DE2, the third depth electrode DE3 and the fourth depth electrode DE4 of the depth electrode set 30D, respectively. The processor would then compare the fifth impedance variations ΔZ5 and the sixth impedance variations ΔZ6 to determine the electrical stimulation control upon the brain tissue 202, and further to control the two middle depth electrode sets 30B, 30C to perform electrical stimulation ST upon nerve fibers surrounding the brain tissue 202. In this embodiment, electrodes in the two middle depth electrode sets 30B, 30C can be applied as the stimulating electrodes for providing the electrical stimulation ST.

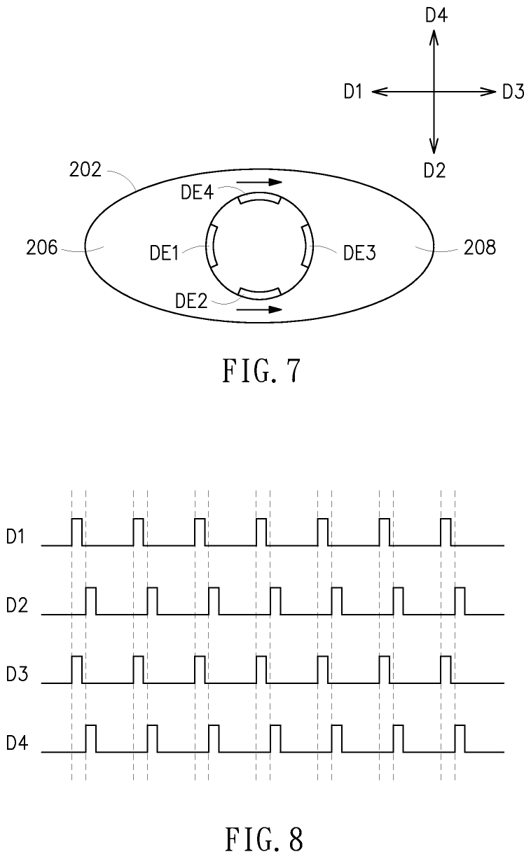

Referring to FIG. 7, the working theory of this disclosure is elucidated. When the human moves back and forth horizontally (i.e., in parallel to the first direction D1 or the third direction D3), the brain tissue 202 would be deformed in the first direction D1 or the third direction D3. For example, if the first lateral brain tissue 206 presents a loose state, then the third lateral brain tissue 208 would demonstrate a compression state. In FIG. 7, the arrow direction is the moving direction of the electrode. Thereupon, instant impedance variations induced by deformations of the first lateral brain tissue 206 and the third lateral brain tissue 208 with respect to the first depth electrode DE1 and the third depth electrode DE3, respectively, during the human movement can be measured (i.e., comparison between the first impedance variation ΔZ1 and the third impedance variation ΔZ3).

Similarly, when the human moves horizontally left and right or vertically up and down, two lateral sides of the brain tissue 202 would be deformed accordingly to induce different instant impedance variations. For example, while in horizontal left and right moving, the second impedance variation ΔZ2 would be compared to the fourth impedance variation ΔZ4. For another example, while in vertical up and down moving, the fifth impedance variation ΔZ5 would be compared to the sixth impedance variation ΔZ6.

Referring to FIG. 8, in a typical design example of a four-direction facing-route synchronous sequential voltage measurement circuit, measurement/switching at four directions (i.e., a first direction D1, a second direction D2, a third direction D3 and a fourth direction D4) are performed to further detect respective impedance variations of voltage.

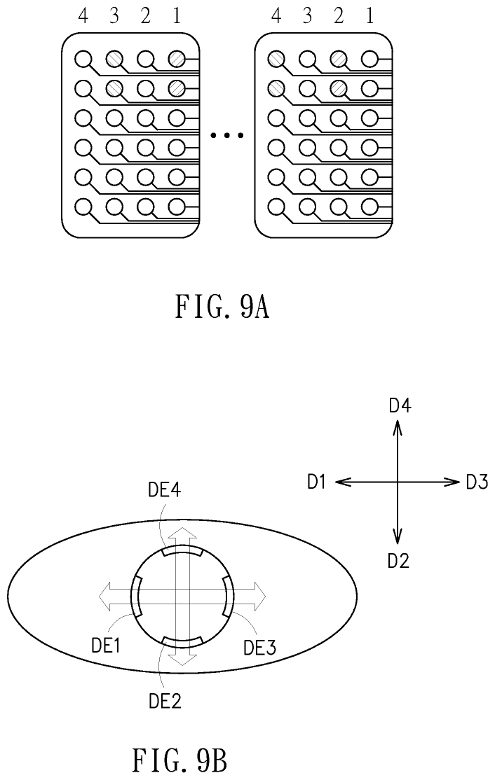

Referring to FIGS. 9A˜9B, 10A˜10B and 11A˜11B, impedance variations at different directions are continuously detected by the multi-channel pairing switches, with a sampling frequency of at least 100 Hz. Number 1, 2, 3 and 4 in FIGS. 9A, 10A and 11A stands for the first direction D1, the second direction D2, the third direction D3 and the fourth direction D4. In these figures, circles in the first row stand for the four flat electrodes (i.e., the first flat electrode FE1, the second flat electrode FE2, the third flat electrode FE3 and the fourth flat electrode FE4) of the flat electrode set 40, circles in the second row stand for the four depth electrodes (i.e., the first depth electrode DE1, the second depth electrode DE2, the third depth electrode DE3 and the fourth depth electrode DE4) of the depth electrode set 30A, circles in the fifth row stand for the four depth electrodes (i.e., the first depth electrode DE1, the second depth electrode DE2, the third depth electrode DE3 and the fourth depth electrode DE4) of the depth electrode set 30D, and circles in the sixth row stand for electrodes of the conduction area 20.

Referring to FIG. 9A and FIG. 9B, while in moving in the first direction D1, the second direction D2, the third direction D3 or the fourth direction D4, impedance variations between the flat electrode set 40 and every directional electrodes of the depth electrode set 30A with respect to deformations of the brain tissue 202 are detected. In FIG. 9A, the electrode channel is switched from the first direction D1 and the third direction D3 to the second direction D2 and the fourth direction D4. In detail, while the electrode channels in the first direction D1 and the third direction D3 are connected, and the first flat electrode FE1 of the flat electrode set 40 and the first depth electrode DE1 of the depth electrode set 30A are connected, then the first impedance variation ΔZ1 can be detected. When the third flat electrode FE3 of the flat electrode set 40 and the third depth electrode DE3 of the depth electrode set 30A are connected, then the third impedance variation ΔZ3 can be measured. As the electrode channel is switched to the second direction D2 and the fourth direction D4, when the electrode channels in the first direction D1 and the third direction D3 are disconnected, the first flat electrode FE1 and the third flat electrode FE3 of the flat electrode set 40 and the first depth electrode DE1 and correspondingly the third depth electrode DE3 of the depth electrode set 30A are all disconnected, and the second flat electrode FE2 of the flat electrode set 40 and the second depth electrode DE2 of the depth electrode set 30A are connected, then the second impedance variation ΔZ2 can be measured. When the fourth flat electrode FE4 of the flat electrode set 40 and the fourth depth electrode DE4 of the depth electrode set 30A are connected, then the fourth impedance variation ΔZ4 can be measured. Thereupon, the processor can evaluate multiple comparisons of the impedance variations to determine the control of the electrical stimulation ST upon the brain tissue 202.

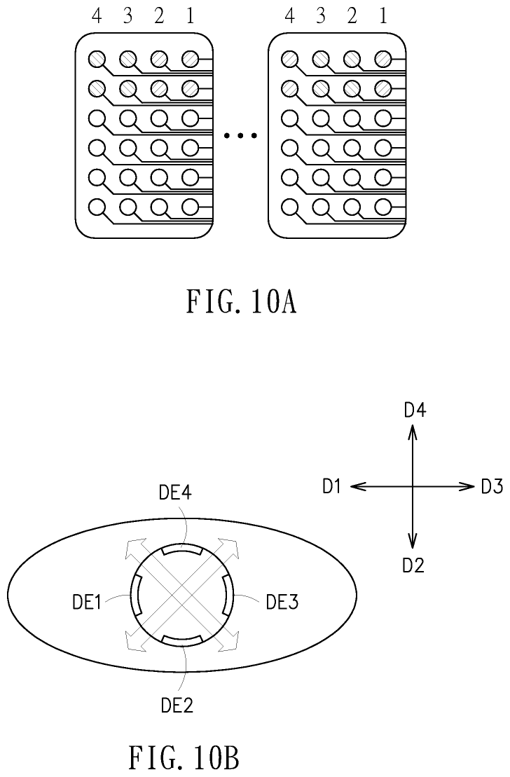

Referring to FIG. 10A and FIG. 10B, while in moving obliquely in the first direction D1, the second direction D2, the third direction D3 or the fourth direction D4, impedance variations between the flat electrode set 40 and every directional electrodes of the depth electrode set 30A with respect to deformations of the brain tissue 202 are detected. In FIG. 10A, the electrode channels of the first and second directions D1, D2 and the third and fourth directions D3, D4 are switched to the electrode channels of the first and fourth directions D1, D4 and the second and third directions D2, D3. In FIG. 10A, the drawing at the left side thereof shows that the four flat electrodes of the flat electrode set 40 and the four depth electrodes of the depth electrode set 30A are connected simultaneously, and thus the processor would compare the impedance variations in first direction D1 and the second direction D2 (i.e., the first impedance variation ΔZ1 and the second impedance variation ΔZ1) with the impedance variations in third direction D3 and the fourth direction D4 (i.e., the third impedance variation ΔZ3 and the fourth impedance variation ΔZ4) to determine the required electrical stimulation ST for the brain tissue 202. On the other hand, the drawing at the right side of FIG. 10A shows that the four flat electrodes of the flat electrode set 40 and the four depth electrodes of the depth electrode set 30A are connected simultaneously, and thus the processor would compare the impedance variations in first direction D1 and the fourth direction D4 (i.e., the first impedance variation ΔZ1 and the fourth impedance variation ΔZ1) with the impedance variations in second direction D2 and the third direction D3 (i.e., the second impedance variation ΔZ2 and the third impedance variation ΔZ3) to determine the required electrical stimulation ST for the brain tissue 202.

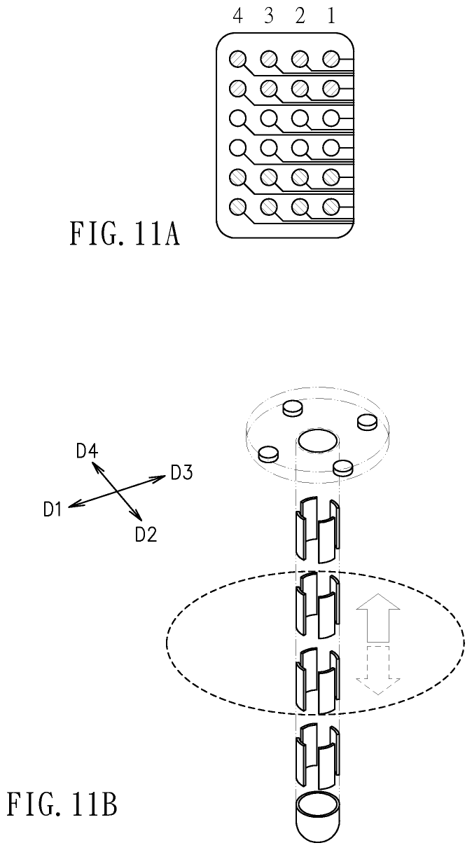

Referring to FIG. 11A and FIG. 11B, while in moving parallel to the axis C10, impedance variations of the depth electrode sets 30A, 30D corresponding to the flat electrode set 40 and the conduction area 20, respectively, with respect to deformations of the brain tissue 202 are detected. In FIG. 11A, the four flat electrodes of the flat electrode set 40 and the four depth electrodes of the depth electrode set 30A, and the electrodes of the conduction area 20 and the four depth electrodes of the depth electrode set 30D are all simultaneously connected, and thus the processor would compare the fifth impedance variations ΔZ5 generated between the flat electrode set 40 and the depth electrode set 30A with the sixth impedance variations ΔZ6 generated between the conduction area 20 and the depth electrode set 30D to determine the required electrical stimulation ST for the brain tissue 202.

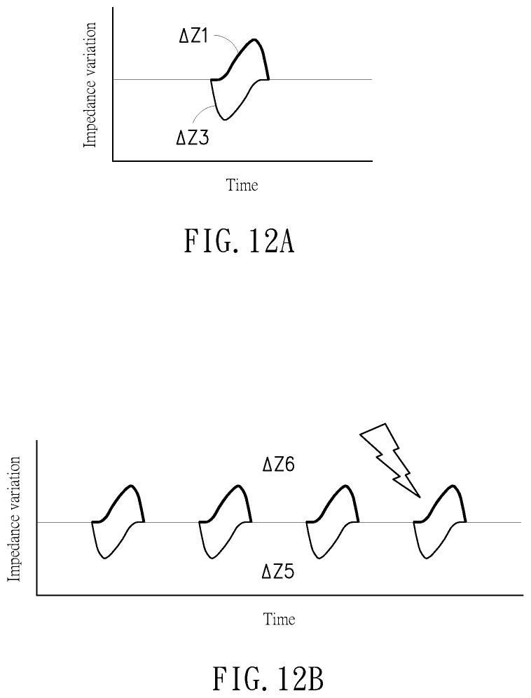

Referring to FIGS. 12A, 12B, in summarizing the aforesaid detection upon the instant impedance variations generated by human movement, the facing-route synchronous sequential voltage measurement circuits and the multi-channel pairing switches, proper electrical stimulation ST at correct timing can be provided through continuous detection upon human movement.

FIG. 12A shows schematically impedance variations while the human rises and tilts forward, from which the detection pattern of horizontal moving corresponding to FIG. 5 and the channel switching pattern corresponding to FIG. 9A can be summarized. In this disclosure, assuming that the first direction D1 is the front direction, when a human rises or tilts forward, the first impedance variation ΔZ1 and third impedance variation ΔZ3 can be referred to FIG. 12A. In one exemplary example, upon when the condition of |ΔZ3−ΔZ1|≥|ΔZ3| is satisfied, the processor would determine that the instant human movement is a human rise or forward tilting, and then a corresponding electrical stimulation mode can be initiated to control the depth electrode sets 30B, 30C to perform the electrical stimulation ST.

FIG. 12B shows schematically impedance variations while the human walks, from which the detection pattern of vertical moving corresponding to FIG. 6 can be summarized. FIG. 12B illustrates schematically the fifth impedance variations ΔZ5 and the sixth impedance variations ΔZ6 while the human walks. In one exemplary example, after the impedance variations ΔZ5, ΔZ6 present three occurrences of a specific characteristic within a preset duration, it is determined that the human is walking, and thus an electrical stimulation mode would be activated to control the depth electrode sets 30B, 30C to perform the corresponding electrical stimulation ST.

It shall be explained that, before the movement monitor sensor provided in this disclosure is implanted into a human brain, corresponding orientations and positions have been understood in advance. Thus, directions of all the electrodes can be realized for controlling and detecting the human movement.

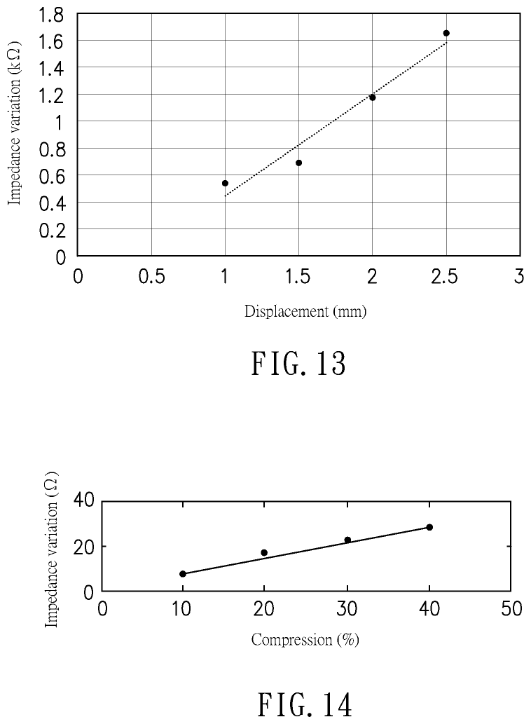

Referring to FIG. 13, a linear relationship between impedance variations and displacements caused by deformations of the brain tissue while the human moves in one direction in accordance with this disclosure is demonstrated schematically. For example, while the human displacement is 1 mm, the corresponding impedance variation is about 0.55 kΩ. When the human displacement is 2 mm, the corresponding impedance variation is about 1.2 kΩ.

Referring to FIG. 14, a linear relationship between impedance variations and compression caused by deformations of the brain tissue while the human moves in one direction in accordance with this disclosure is demonstrated schematically. For example, when the brain-tissue compression rate is 10%, the corresponding impedance variation is about 8Ω. When the brain-tissue compression rate is 40%, the corresponding impedance variation is about 30Ω.

With FIG. 13 and FIG. 14, the applicability that, in accordance with this disclosure, detection of impedance variations corresponding to the human displacements and brain-tissue compression can be utilized to determine the electrical stimulation upon the human brain has been confirmed.

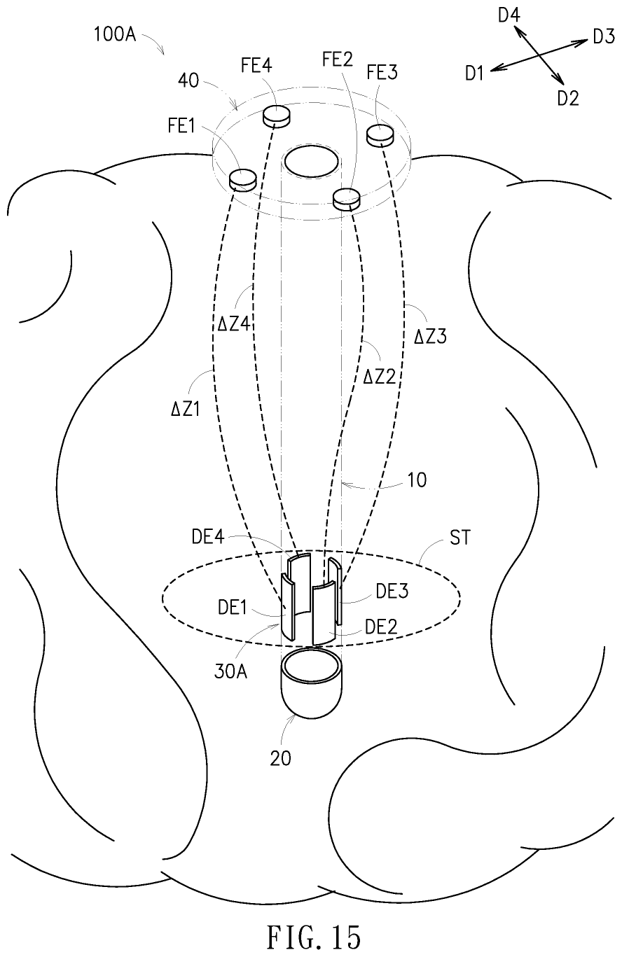

Referring to the embodiment shown in FIG. 15, the movement monitor sensor 100A includes a body 10, a conduction area 20, a depth electrode set 30A and a flat electrode set 40.

As shown in FIG. 15, when the human moves in the first direction D1 and the third direction D3 (for example, moving horizontally back and forth), the first flat electrode FE1 and the first depth electrode DE1 of the depth electrode set 30A would generate a first impedance variation ΔZ1, and the third flat electrode FE3 and the third depth electrode DE3 of the depth electrode set 30A would generate a third impedance variation ΔZ3. By having the processor to evaluate the first impedance variation ΔZ1 and the third impedance variation ΔZ3 for determining the corresponding electrical stimulation for the depth electrode set 30A to control the brain tissue 202, then the electrical stimulation ST can be applied to the nerve fibers surrounding the brain tissue 202. In this embodiment, the electrodes in the depth electrode set 30A can be used as the stimulating electrodes for providing the corresponding electrical stimulation ST.

Similarly, when the human moves in the second direction D2 or in the fourth direction D4 (for example, moving horizontally left and right), the second flat electrode FE2 and the second depth electrode DE2 of the depth electrode set 30A would generate a second impedance variation ΔZ2, and the fourth flat electrode FE4 and the fourth depth electrode DE4 of the depth electrode set 30A would generate a fourth impedance variation ΔZ4. The processor would then compare the second impedance variation ΔZ2 to the fourth impedance variation ΔZ4 so as to form the electrical stimulation control upon the brain tissue 202, and further to control the depth electrode set 30A to perform the corresponding electrical stimulation ST upon the nerve fibers surrounding the brain tissue 202.

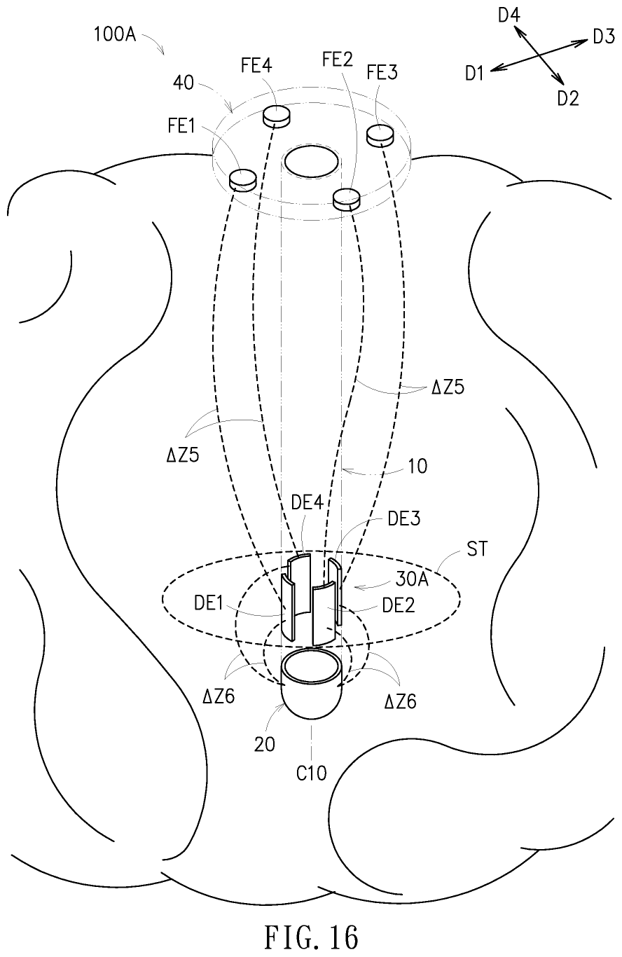

Referring to FIG. 16, when the human moves in parallel to the axis C10 (for example, moving vertically up and down), the first flat electrode FE1, the second flat electrode FE2, the third flat electrode FE3 and the fourth flat electrode FE4 would react individually with the first depth electrode DE1, the second depth electrode DE2, the third depth electrode DE3 and the fourth depth electrode DE4 of the depth electrode set 30A, respectively, to generate corresponding fifth impedance variations ΔZ5. In addition, the conduction area 20 would react with each of the first depth electrode DE1, the second depth electrode DE2, the third depth electrode DE3 and the fourth depth electrode DE4 of the depth electrode set 30D so as to generate a corresponding sixth impedance variation ΔZ6. After the processor evaluates the fifth impedance variation ΔZ5 and the sixth impedance variation ΔZ6, the electrical stimulation control upon the brain tissue 202 can be thus generated for controlling the depth electrode set 30A, such that corresponding electrical stimulation ST can be executed upon the nerve fibers surrounding the brain tissue 202.

It shall be noted that, though the embodiments of FIG. 1 and FIG. 15 have four depth electrode sets 30A˜30D and one depth electrode set 30A, respectively, yet, according to this disclosure, even an embodiment including one single depth electrode set can achieve the design goal of this disclosure. Namely, no matter if one or four said depth electrode sets are furnished, the working theory for realizing the human movement through the detection of the instant impedance variations generated thereby in accordance with this disclosure prevails to the facing-route synchronous sequential voltage measurement and the multi-channel pairing switches. This disclosure can be also applied to detection of continuous human movements for determining relevant electrical stimulation at correct timing. In addition, the depth electrode set of this disclosure can adopt two, three or more than four sets of the depth electrode sets, but preferably depending on practical requirements.

In summary, the movement monitor sensor for the electrical stimulation control provided in this disclosure, the flat electrodes, the depth electrodes and the conduction area are utilized to measure instant impedance variations caused by deformations of brain tissue during human movement, such that any instant state change of the human, moving or not, can be realized to evaluate the timing for activating the electrical stimulation. Thereupon, those patients who are used to present poor responses against the conventional continuous electrical stimulation would be given the electrical stimulation only at the switching points in the gait cycle (such as a posture change from sitting to standing, at turning, or at a heal strike or a liftoff), and thus patient mobility in walking can be effectively improved.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. A movement monitor sensor, disposed in a human brain, suitable to connect a processor, comprising:
a body, having an axis and two axial ends along the axis and opposite to each other;
a conduction area, disposed at one of the two axial ends, connected with a conductive wire;
at least one depth electrode set, including four depth electrodes disposed on a surface of the body by surrounding the axis, the four depth electrodes being distributed to four directions, each of the four depth electrodes being connected with a first wire; and
a flat electrode set, including a substrate and four flat electrodes, the substrate having oppositely a first surface and a second surface, the second surface being disposed at one of the two axial ends opposite to the conduction area, the four flat electrodes being disposed at the substrate by surrounding the axis to correspond individually the four directions, each of the four flat electrodes being connected with a second wire;
wherein the conductive wire, the first wires and the second wires are individually connected electrically with the conduction area, the depth electrodes and the flat electrodes; wherein, when a movement of a human changes, the processor evaluates impedance variations generated by the at least one depth electrode set, the flat electrode set and/or the conduction area to determine a corresponding electrical stimulation control upon brain tissue of the human.

2. The movement monitor sensor of claim 1, wherein the substrate has a central through hole extending in parallel to the axis, and the four flat electrodes surround the central through hole.

3. The movement monitor sensor of claim 2, wherein the first wires, the second wires and the conductive wire are individually extended from the body to an exterior via the central through hole of the substrate.

4. The movement monitor sensor of claim 1, wherein the second surface of the substrate is furnished with a micro structure.

5. The movement monitor sensor of claim 4, wherein the micro structure protrudes out of the substrate by a height equal to or greater than 50 μm.

6. The movement monitor sensor of claim 1, wherein the four direction includes a first direction, a second direction, a third direction and a fourth direction; the first direction, the second direction, the third direction and the fourth direction are individually perpendicular to the axis, centered at the axis by equal angular spacing; the first direction and the third direction are located oppositely with respect to the axis, the second direction and the fourth direction are located oppositely with respect to the axis, and the first direction is located between the second direction and the fourth direction; the four depth electrodes include a first depth electrode, a second depth electrode, a third depth electrode and a fourth depth electrode corresponding to the first direction, the second direction, the third direction and the fourth direction, respectively; and, the four flat electrodes include a first flat electrode, a second flat electrode, a third flat electrode and a fourth flat electrode facing the first direction, the second direction, the third direction and the fourth direction, respectively.

7. The movement monitor sensor of claim 6, wherein, when the human moves in the first direction or the third direction, a first impedance variation is generated between the first flat electrode and the first depth electrode of the depth electrode set, a third impedance variation is generated between the third flat electrode and the third depth electrode of the depth electrode set, and the processor compares the first impedance variation to the third impedance variation so as to determine the corresponding electrical stimulation control upon the brain tissue of the human.

8. The movement monitor sensor of claim 6, wherein, when the human moves in the second direction or the fourth direction, a second impedance variation is generated between the second flat electrode and the second depth electrode of the depth electrode set, a fourth impedance variation is generated between the fourth flat electrode and the fourth depth electrode of the depth electrode set, and the processor compares the second impedance variation to the fourth impedance variation so as to determine the corresponding electrical stimulation control upon the brain tissue of the human.

9. The movement monitor sensor of claim 6, wherein, when the human moves in parallel to the axis, a fifth impedance variation is generated between each of the first flat electrode, the second flat electrode, the third flat electrode and the fourth flat electrode and corresponding one of the first depth electrode, the second depth electrode, the third depth electrode and the fourth depth electrode of the depth electrode set, respectively, a sixth impedance variation is generated between the conduction area and each of the first depth electrode, the second depth electrode, the third depth electrode and the fourth depth electrode of the depth electrode set, and the processor compares the fifth impedance variation to the sixth impedance variation so as to determine the corresponding electrical stimulation control upon the brain tissue of the human.

10. The movement monitor sensor of claim 1, wherein the first surface of the substrate is furnished with a fixing part to be disposed at a head bone of the human, and the brain tissue is located under the head bone.

11. The movement monitor sensor of claim 10, wherein the fixing part has a channel for allowing the first wires, the second wires and the conductive wire to extend thereinside and to pass therethrough for further protruding out of the fixing part to exterior of the head bone.

12. The movement monitor sensor of claim 1, wherein the substrate is shaped to be a ring disc having a maximum diameter equal to or less than 10 mm.

13. The movement monitor sensor of claim 1, wherein the substrate is made of a silicone or a thermoplastic polyurethane (TPU).

14. The movement monitor sensor of claim 1, wherein the at least one depth electrode set includes four said depth electrode sets, the four depth electrode sets are disposed at the body in parallel to the axis, the four depth electrodes of each of the four depth electrode sets are corresponding to the four directions, part of the four depth electrode sets are served as stimulating electrodes for performing electrical stimulation upon the brain tissue of the human.
